(19) United States
(12) Patent Application Publication
(10) Pub. No.: US 2011/0268821 A1
(43) Pub. Date: Nov. 3, 2011

(54) EXTRACTING AN EXTRACT SUBSTANCE FROM A RAW MATERIAL

(75) Inventor: Alexey Sheptitskiy, Darlinghurst (AU)

(73) Assignee: DDF (AUSTRALIA) PTY LTD, Darlinghurst (AU)

(21) Appl. No.: 12/739,132

(22) PCT Filed: Oct. 30, 2008
(86) PCT No.: PCT/AU08/01599

§ 371 Date: Apr. 21, 2010

(30) Foreign Application Priority Data
Oct. 30, 2007 (AU) 2007905912
Nov. 16, 2007 (AU) 2007906287

(51) Int. Cl. A61K 36/00; A23L 1/00; B01J 8/00; A23P 1/00

(52) U.S. Cl. 424/725; 426/431; 422/187

(57) ABSTRACT

The present invention relates broadly to the extraction of an extract substance from a raw material. The extract substance may have application in the food, cosmetics, insecticide or other industries. The raw material may be in the form of vegetable or fruit matter or waste. The extract substance is to be in the form of a concentrated food (for humans or animals) or cosmetic including but not limited to aromatic substances.

### FIELD OF THE INVENTION

The present invention relates broadly to a method and apparatus for extracting an extract substance from a raw material, such as vegetable or fruit wastes.

### BACKGROUND OF THE INVENTION

It is known to extract concentrated food or cosmetic products from raw materials using solvents or liquefied gases, such as liquid carbon dioxide. This process typically involves passing the liquid CO₂ through the raw material (eg vegetable waste) to absorb or dissolve an extract (eg healthy components of biologically active substances) which is later recovered by distillation. This prior art technique can take as long as 3 to 6 hours and is performed at a sub-critical temperature and pressure for the liquid CO₂.

### SUMMARY OF THE INVENTION

According to one aspect of the present invention there is provided a method of extracting an extract substance from a raw material, the method comprising the steps of:
exposing the raw material to gaseous carbon dioxide;
mixing the raw material with liquid carbon dioxide at a temperature below its critical temperature to form a raw material mixture where the extract substance at least in part dissolves into the liquid carbon dioxide; and
agitating the raw material mixture through a rotary grinding and cavitation pump for fracturing of the raw material into relatively fine particles including the extract substance.

Preferably the method also comprises the pretreatment step of degassing the raw material.

Preferably the step of exposing the raw material to gaseous carbon dioxide involves passing gaseous carbon dioxide through the raw material which is previously degassed. Alternatively the step of exposing the raw material to gaseous carbon dioxide involves pressurising the raw material with the gaseous carbon dioxide.

Preferably the step of mixing the raw material with liquid carbon dioxide is performed under and close to the subcritical pressure of carbon dioxide for extraction of the extract substance. More preferably the raw material together with the liquid carbon dioxide are sufficiently agitated by the rotary grinding and cavitation pump to provide cavitation for increased size reduction.

Preferably the step of mixing is performed just under the carbon dioxide critical temperature to promote the raw material mixture dissolution into the liquid carbon dioxide. More preferably the mixing and extraction are performed under 15° C. to 25° C. which is sufficient to promote this dissolution.

According to another aspect of the invention there is provided a method of extracting an extract substance from a raw material, the method comprising the steps of:
adding liquid carbon dioxide to the raw material to form a raw material mixture within an extractor; and
fracturing of the raw material into relative fine particles including the extract substance by recirculating the raw material mixture through a rotary grinding and cavitation pump located outside the extractor.

Preferably the method also comprises the step of combining the fine particles and additional liquid carbon dioxide to form a concentrated solution of the extract substance.

Preferably the method also comprises the step of adding gaseous carbon dioxide to the raw material prior to the addition of Liquid carbon dioxide. More preferably the gaseous carbon dioxide is added in two (2) stages with the pressure after the first stage being about 50% of the pressure after the second stage.

Preferably the step of adding liquid carbon dioxide to the raw material increases the working pressure of the raw material mixture to around 60 Bar.

Preferably the step of mixing the raw material with liquid carbon dioxide is performed under and close to the subcritical pressure of carbon dioxide at around 60 Bar. More preferably cavitation of the mixture is provided by reducing the pressure of the raw material mixture in the rotary grinding and cavitation pump. Still more preferably cavitation of the mixture is provided by high agitation grinding via the rotary grinding device which is effective in extraction of the extract substance.

According to a further aspect of the invention there is provided an apparatus for extracting an extract substance from a raw material, the apparatus comprising:
a mixing chamber being adapted to contain a pressurised mixture of liquid carbon dioxide and the raw material;
a rotary grinding and cavitation pump operatively coupled to and located outside the mixing chamber to receive a recirculatory flow of the pressurised raw material mixture for fracturing of the raw material into relatively fine particles including the extract substance.

Preferably the apparatus also comprises an extractor operatively coupled to the rotary grinding and cavitation pump to receive the fine particles and combine them with additional liquid carbon dioxide to form a concentrated solution of the extract substance.

Preferably the rotary grinding and cavitation pump is effective in reducing the pressure of the mixture to provide cavitation and fracturing of the raw material including the extract substance into relatively fine particles.

Preferably the apparatus also comprises a heat exchanger operatively coupled to the extractor or the rotary grinding and cavitation pump for vaporisation of at least some of the liquid carbon dioxide from the mixture to further concentrate the extract substance. More preferably the heat exchanger is a vaporiser.

Preferably the apparatus also comprises a carbon dioxide condenser being operatively coupled to the mixing chamber and/or the extractor to provide a supply of liquid carbon dioxide. More preferably the condenser includes a liquid carbon dioxide collector or storage vessel elevated above the chamber and/or extractor.

### BRIEF DESCRIPTION OF THE DRAWINGS

In order to achieve a better understanding of the nature of the present invention a preferred embodiment of a method and apparatus for extracting an extract substance from a raw material will now be described, by way of example only, with reference to the accompanying drawings in which:

FIG. 1 is a schematic flow diagram of an apparatus according to one embodiment of the invention;

FIG. 2 is a schematic flow diagram of an apparatus according to another embodiment of the invention;

FIG. 3 is a part sectional view of a rotary grinding device taken from the apparatus of FIG. 2; and

FIG. 4 is a pressure-temperature phase diagram for carbon dioxide.

### DETAILED DESCRIPTION OF THE PREFERRED EMBODIMENT

The present invention relates generally to the extraction of an extract substance from a raw material. The extract substance may have application in the food, cosmetics, insecticide or other industries. The raw material may be in the form of vegetable or fruit matter or waste such as orange peels, lemon myrtle, tea tree, grapes, apple seeds, kiwi fruit, black paper or other materials. The extract substance is typically in the form of a concentrated food (for humans or animals) or cosmetic including but not limited to aromatic substances.

The general steps involved in the methodology of one embodiment of this invention are as follows:

1. The raw material such as plants' leaves and seeds are agitated via a gaseous carbon dioxide stream;

2. The raw material is mixed with liquid carbon dioxide at a sub-critical temperature to form a raw material mixture;

3. The raw material mixture is pressurised by for example heating of the liquid CO₂ to a sub-critical temperature of around 15° C. to 25° C. at around 60 Bar;

4. The pressurised raw material mixture is released for a rapid reduction in pressure to provide cavitation and fracturing of the raw material into relatively fine particles including the extract substance;

5. The relatively fine particles are combined with additional liquid CO₂ to form a concentrated solution of the extract substance.

The raw material generally includes relatively large particles which may be milled or crushed to a reduced particle size of between around 0.1 to 0.35 millimetres.

The CO₂ gas is in step 1 charged for a period of around 5 minutes and the mixing of the raw material with liquid CO₂ in step 2 is performed for around 15 minutes. The CO₂ gas in step 1 passes through the raw material which is previously degassed, and all the mixing of liquid CO₂ in step 2 is performed under and close to its subcritical pressure at around 60 Bar.

In steps 2 and 3 while adding liquid CO₂ to vegetative raw material it penetrates through cells' membranes and impregnates them dissolving substances inside the cells. In step 4 when the raw material is impregnated with liquid CO₂ the pressure decreases and liquid CO₂ turns into a gaseous condition. The CO₂ volume dramatically increases (e.g. 300-500 times) that leads to internal explosions of cells, i.e. crushing the raw material by cell explosions as liquid CO₂ converts into a gaseous condition.

The extraction process at step 5 may vary in time from 90 to 180 minutes.

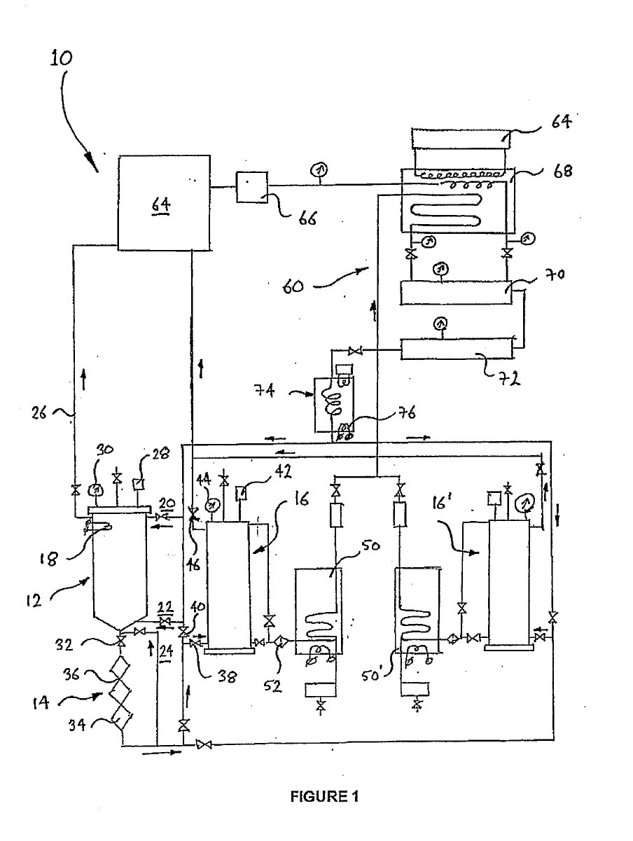

The core components of the apparatus 10 for implementing this extraction methodology and process are shown in FIG. 1.

The charging, mixing and pressurisation operations of steps 1 to 3 are performed in the mixing chamber 12. The cavitation device or mass exchange device 14 provides the cavitation and fracturing of step 4 and an extractor 16 enables the extraction of step 5.

The mixing chamber 12 includes a heater 18 and upper and lower liquid CO₂ inlets 20 and 22, respectively. The chamber or vessel 12 also includes a CO₂ gas inlet 24 at its base. The pressure vessel or chamber 12 also includes a CO₂ gas outlet 26 together with a pressure relief valve 28 and pressure transducer 30 at its upper regions.

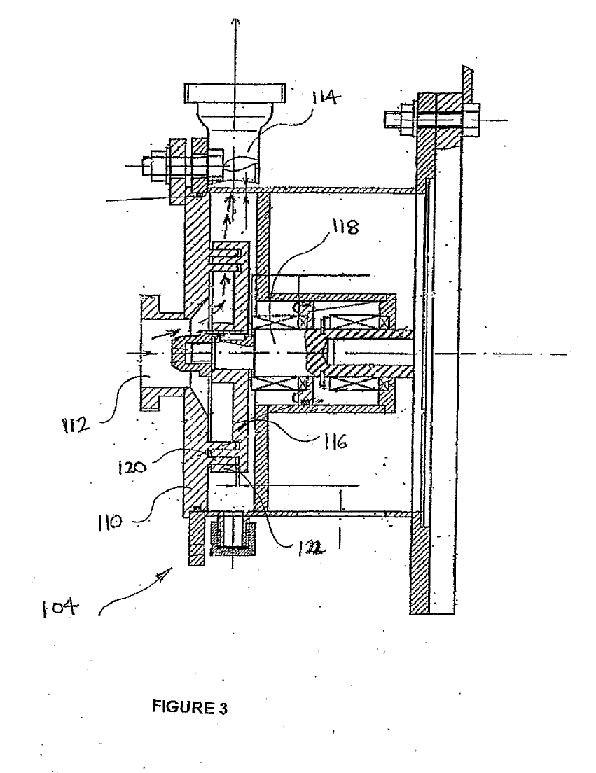

The rotary device 104 may be located outside the extractor 102 or preferably submerged within the extractor 102. The rotary device 104 of this embodiment is best shown in FIG. 3 and is similar in general construction and flow to a centrifugal pump. The rotary device 104 includes a stator 110 defining an axial fluid inlet 112 and at least one radial outlet such as 114. The rotary device 104 also includes a rotor 116 connected to a shaft 118 having appropriate bearings, seals and an associated drive system. The stator 110 and rotor 116 have opposing and mating circular rows of teeth. In this embodiment both the stator 110 and the rotor 116 have two rows of inter-meshed teeth such as 120 and 122, respectively. The number of teeth in each of the rows may vary from 15 to 40, depending on the size of the rotary device and its grinding application.

As described in the context of the methodology applicable to the apparatus 10 of FIG. 1, the liquid CO₂ penetrates the cell membranes of the vegetative raw material thereby impregnating it. The rotary device 104 is in the form of a rotary grinding and cavitation pump which has caves or apertures located at or adjacent the inter-meshed teeth 120/122 of the stator 110 and the rotor 116. When the rotor 116 rotates its apertures are moving besides the stator 110 apertures. In this process the external volume between rotor's and stator's caves increases the impregnated raw material liquid CO₂ boils up breaking cells from the inside. This leads to a dramatic acceleration of dissolving of internal food and health substances into liquid CO₂. The raw material crushing occurs by not only mechanical action of the grinding teeth but also by a dramatic increase in the external volume between the rotor's and the stator's apertures. The rotary grinding and cavitation pump also has the function of a centrifugal pump which creates a stream of mixture of liquid CO₂ and crushed raw material. Raw material washes by the liquid CO₂ changing phase and accelerating the dissolution of substances during the process of extraction.

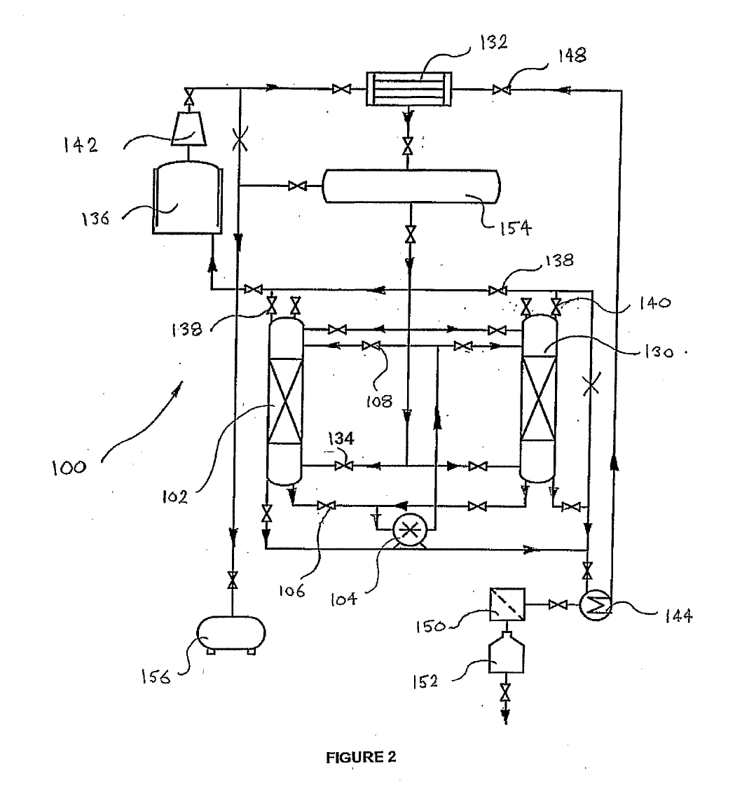

In the flow diagram of FIG. 2 it can be seen that the extractor 102 is one of a pair of extractors 102 and 130. The apparatus 100 also includes a condenser 132 which supplies the liquid CO₂ for charging the extractors 102 and 130 via respective valves such as 134.

The apparatus 100 also comprises a gas holder 136 which following the extraction process and opening of the CO₂ gas valves such as 138 and 140 receives gaseous CO₂ from the extractors such as 102 and 130. A compressor 142 provides compressed CO₂ gas to the condenser 132.

In this example the concentrated solution of the extract substance following its extraction from the raw material mixture passes through a heat exchange in the form of vaporiser 144. This separates any residual liquid carbon dioxide from the concentrate and returns it in its gaseous form to the condenser 132 via carbon dioxide gaseous return valve 148. Additionally, the apparatus 100 includes a filter 150 and extract collector 152 located downstream of the vaporiser 144.

The apparatus 100 additionally comprises a liquid CO₂ collector 154 positioned between the condenser 132 and the extractors 102 and 130. The collector 154 is also connected to a liquid CO₂ cistern 156 to accommodate excessive liquid CO₂ in the system.

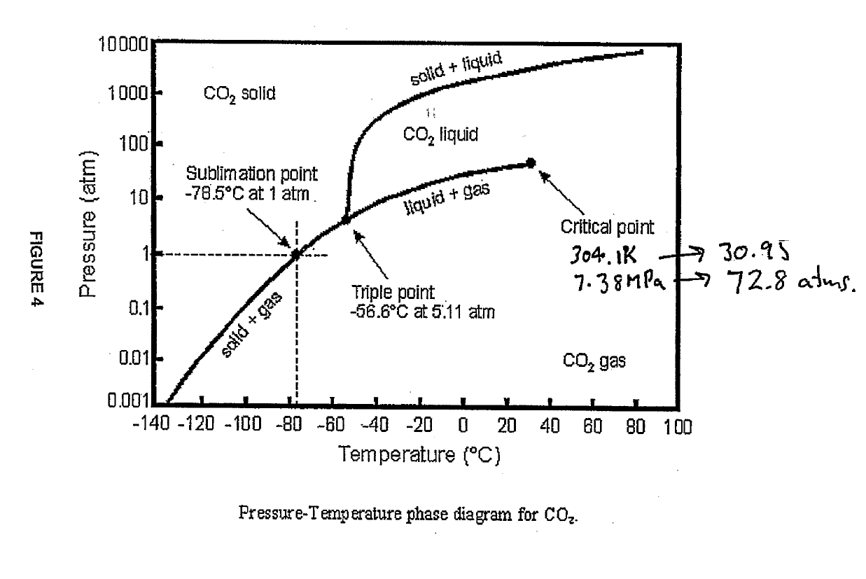

FIG. 4 is a pressure/temperature phase diagram for CO₂ showing the operating pressure/temperature in the preferred method of this invention. It can be seen that the mixing of the raw material and CO₂ is performed at a sub-critical temperature, where the critical point for CO₂ is shown at a pressure and temperature of around 73 Bar (or atmospheres) and 30° C.

The subsequent pressurisation of the raw material mixture is still performed at sub-critical temperature and pressure conditions. It is understood that the liquid CO₂ penetrates or migrates inside the raw material, such as the plant cell. As explained earlier, the rapid pressure reduction and cavitation, the liquid CO₂ contained within the raw material or plant cell evaporates or is at least in part vaporised thereby fracturing or rupturing the raw material or plant cell evaporates or is at least in part vaporised thereby fracturing or rupturing the raw material or plant cell into relatively fine particles including the extract substances.

It will be apparent to those skilled in the art that the method and apparatus for extracting an extract substance from a raw material has the following advantages:
1. the process is more efficient that the prior art technique which requires an extended period wherein the raw material is exposed to liquid CO₂;
2. the operating pressures are less than the prior art which requires up to 1,000 Bar;
3. the apparatus and plant required are less expensive because of the lower temperature and pressure operating conditions; and
4. the process is more efficient in recovering relatively high levels of the extract substance from the raw material.

Those skilled in the art will appreciate that the invention described herein is susceptible to variations and modifications other than those specifically described. For example, the specific construction of the apparatus may vary from that described provided it does not depart greatly from the core unit operations disclosed in this specification. Similarly, the pressure/temperature parameters may vary from these specific values described in the specification. All such variations and modifications are to be considered within the scope of the present invention the nature of which is to be determined from the foregoing description.

1-23. (canceled)

24. A method of extracting an extract substance from a raw material, the method comprising the steps of:
exposing the raw material to gaseous carbon dioxide;
mixing the raw material with liquid carbon dioxide at a temperature below its critical temperature to form a raw material mixture where the extract substance at least in part dissolves into the liquid carbon dioxide; and
agitating the raw material mixture through a rotary grinding and cavitation pump for fracturing of the raw material into relatively fine particles including the extract substance.

25. The method as defined in claim 24 also comprising the pretreatment step of degassing the raw material.

26. The method as defined in claim 25 wherein the step of exposing the raw material to gaseous carbon dioxide involves passing gaseous carbon dioxide through the raw material which is previously degassed.

27. The method as defined in claim 24 wherein the step of exposing the raw material to gaseous carbon dioxide involves pressurising the raw material with the gaseous carbon dioxide.

28. The method as defined in claim 24 wherein the step of mixing the raw material with liquid carbon dioxide is performed under and close to the subcritical pressure of carbon dioxide for extraction of the extract substance.

29. The method as defined in claim 24 wherein the raw material together with the liquid carbon dioxide are sufficiently agitated by the rotary grinding and cavitation pump to provide cavitation for increased size reduction.

30. The method as defined claim 24 wherein the step of mixing is performed just under the carbon dioxide critical temperature to promote the raw material mixture dissolution into the liquid carbon dioxide.

31. The method as defined in claim 30 wherein the step of mixing and extraction are performed under 15° C. to 25° C. which is sufficient to promote this dissolution.

32. A method of extracting an extract substance from a raw material, the method comprising the steps of:
adding liquid carbon dioxide to the raw material to form a raw material mixture within an extractor;
fracturing of the raw material into relative fine particles including the extract substance by recirculating the raw material mixture through a rotary grinding and cavitation pump located outside the extractor.

33. The method as defined in claim 32 also comprising the step of combining the fine particles and additional liquid carbon dioxide to form a concentrated solution of the extract substance.

34. The method as defined in claim 32 also comprising the step of adding gaseous carbon dioxide to the raw material prior to the addition of liquid carbon dioxide.

35. The method as defined in claim 34 wherein the gaseous carbon dioxide is added in two (2) stages with the pressure after the first stage being about 50% of the pressure after the second stage.

36. The method as defined in claim 24 wherein the step of adding liquid carbon dioxide to the raw material increases the working pressure of the raw material mixture to around 60 Bar.

37. The method as defined in claim 24 wherein the step of mixing the raw material with liquid carbon dioxide is performed under and close to the subcritical pressure of carbon dioxide at around 60 Bar.

38. The method as defined in claim 24 wherein cavitation of the mixture is provided by reducing the pressure of the raw material mixture in the rotary grinding and cavitation pump.

39. The method as defined in claim 38 wherein cavitation of the mixture is provided by high agitation grinding via the rotary grinding device which is effective in extraction of the extract substance.

40. An apparatus for extracting an extract substance from a raw material, the apparatus comprising:
a mixing chamber being adapted to contain a pressurised mixture of liquid carbon dioxide and the raw material;
a rotary grinding and cavitation pump operatively coupled to and located outside the mixing chamber to receive a recirculatory flow of the pressurised raw material mixture for fracturing of the raw material into relatively fine particles including the extract substance.

41. The apparatus as defined in claim 40 also comprising an extractor operatively coupled to the rotary grinding and cavitation pump to receive the fine particles and combine them with additional liquid carbon dioxide to form a concentrated solution of the extract substance.

42. The apparatus as defined in claim 41 wherein the rotary grinding and cavitation pump is effective in reducing the pressure of the mixture to provide cavitation and fracturing of the raw material including the extract substance into relatively fine particles.

43. The apparatus as defined in claim 42 also comprising a heat exchanger operatively coupled to the extractor or the rotary grinding and cavitation pump for vaporisation of at least some of the liquid carbon dioxide from the mixture to further concentrate the extract substance.

44. The apparatus as defined in claim 43 wherein the heat exchanger is a vaporiser.

45. The apparatus as defined in claim 41 also comprising a carbon dioxide condenser being operatively coupled to the mixing chamber and/or the extractor to provide a supply of liquid carbon dioxide.

46. The apparatus as defined in claim 45 wherein the condenser includes a liquid carbon dioxide collector or storage vessel elevated above the chamber and/or extractor.
